(19) United States
(12) Patent Application Publication
(10) Pub. No.: US 2002/0198481 A1
(43) Pub. Date: Dec. 26, 2002

(54) Orthopedic adjustment mechanism for an orthopedic brace

(76) Inventor: Jay Humphries, Covina, CA (US)

Correspondence Address: CHRISTIE, PARKER & HALE, LLP, P.O. BOX 7068, PASADENA, CA, 91109-7068, US

(21) Appl. No.: 10/057,109

(22) Filed: Oct. 18, 2001

Related U.S. Application Data
(60) Provisional application No. 60/299,496, filed on Jun. 20, 2001.

(51) Int. Cl. A61F 5/00

(52) U.S. Cl. 602/23

(57) ABSTRACT

An adjustment device for attachment to an orthopedic brace having a connecting plate upon which an inner disk is secured. The inner disk contains a patterned section which forms an interlocking coupling with a patterned portion of an outer disk to releasably interlock the inner disk to the outer disk.

### CROSS REFERENCE TO RELATED APPLICATION

This application claims the benefit under 35 U.S.C. §119(e) of U.S. Provisional Application No. 60/299,496, filed on Jun. 20, 2001.

### FIELD OF THE INVENTION

This invention relates to an adjustment device for an orthopedic brace. More particularly, this invention relates to an adjustment device for an orthopedic brace that allows for the orthopedic brace to be adjusted both laterally and angularly to a desired position, while comprising an attachment assembly that minimizes lateral and angular movements after the desired position of the orthopedic brace has been obtained.

### BACKGROUND OF THE INVENTION

Current orthopedic braces are manufactured in standard unisex sizes: small, medium, large, etc. However, in order for these orthopedic braces to be custom fit to various individual users, these braces must provide for some adjustability to compensate for the variation in the bodily dimensions of the various users even after the users have been subdivided into small, medium, large, etc. categories.

Current hip braces, for example, provide for angular as well as lateral adjustment. The angular adjustment is provided to compensate for the anatomical differences between male and female patients. The pelvic structure of a typical female is angled more widely with respect to vertical than the pelvic structure of the typical male. The lateral adjustment is provided to compensate for the differences in the waist sizes of the various users of the hip braces.

A current hip brace comprises a semi-rigid plate to which two hip brace shells are attached. After the hip brace shells have been placed to a desired position on the hips of the intended user, the left shell is attached to the plate by screwing an outer disk to an inner disk. A compression force produced by screwing the disks together holds the left shell to the plate. However, in order to screw the disks together, the user of the brace, who has typically just undergone hip surgery, must roll on his or her side in order for the person adjusting the brace to be able to reach behind the user's back, where the plate is located, and insert, then rotate, an alien wrench into a hexagon hole located on the face of the outer disk. Sitting in this position typically places the user of the brace in a considerable amount of pain, especially if the user has just undergone hip surgery.

In addition to the compression forces exerted on the left shell and the plate by the inner and outer disks, amplified frictional forces help reduce unwanted angular or lateral movement between the left shell and the plate once the desired position of the brace has been obtained. The amplified frictional forces are produced by securing textured surfaces, such as sandpaper, to an outside portion of the left shell and to an inside portion of the plate. The description above applies equally to the relationship between the right shell and the plate. However, this arrangement does not adequately prevent relative movement between the shells and the plate when large forces are applied to the brace or when the brace is used for an extended period of time.

Consequently, an improved adjustment device for orthopedic braces is needed to address the problems of the prior art.

### SUMMARY OF THE INVENTION

The present invention addresses the above-referenced problems by providing an inner disk having a patterned section which mates with a patterned portion of an outer disk to releasably interlock the inner disk to the outer disk, and thereby minimize relative motion between an orthopedic brace and the adjustment device of the present invention when the orthopedic brace is adjusted to a desired angular and lateral position with respect to the adjustment device.

In addition, the inner disk and the outer disk are compressed together by securing an attachment knob to the inner disk. Avoiding the problem of the prior art, the attachment knob can be secured to the inner disk by manually turning an ergonomically designed portion of the attachment knob so that a doctor can perform each of the steps of loosening the attachment knobs, adjusting the orthopedic brace with respect to the adjustment device, and tightening the attachment knobs, all with only a minimal movement on the part of the user.

Other objects, advantages and novel features of the present invention will become apparent from the following detailed description of the invention when considered in conjunction with the accompanying drawings.

### DESCRIPTION OF THE DRAWINGS

FIG. 1 is an exploded perspective view of an adjustment device for an orthopedic brace of the present invention;

FIG. 2 is a perspective view of an inner surface of an inner disk. This view shows one embodiment of a patterned section on the inner disk which is used to interlock with a pattered portion of an outer disk;

FIG. 3 is a perspective view of a top surface of an attachment knob. This view shows one embodiment of an ergonomic design of the attachment knob which allows the knob to be manually turned without the need for a tool;

FIG. 4 is a perspective view of a partial assembly of the adjustment device of FIG. 1; and

FIG. 5 is a perspective view of an orthopedic hip device utilizing the adjustment device of FIG. 1.

### DETAILED DESCRIPTION OF THE INVENTION

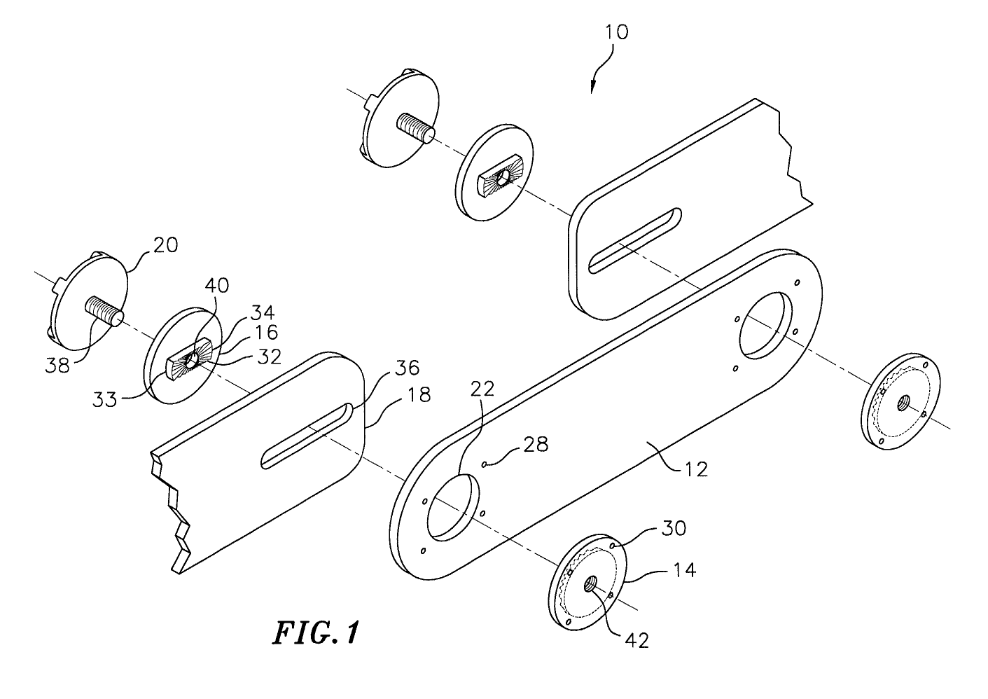

An adjustment device 10 for an orthopedic brace of the present invention is shown in FIGS. 1-3. The adjustment device 10 comprises a semi-rigid connecting plate 12 to which an 15 orthopedic brace, partially shown in FIG. 1 as an orthopedic brace attachment arm 18, is releasably locked into position utilizing an inner disk 14, and an outer disk 16. The inner disk 14 is secured to the plate 12 and forms an interlocking coupling with the outer disk 16. The outer disk 16 mates with the orthopedic brace attachment arm 18 and is secured to the inner disk 14 by use of an attachment knob 20.

In one embodiment, the plate 12 comprises an opening 22, such as a circular opening and the inner disk 14 comprises a patterned section 24 (shown in FIG. 2). The patterned section 24 can, for example, protrude from the inner disk 14 and have a shape similar to the shape of the plate opening 22 such that the patterned section 24 fits snugly into the plate opening 22. The inner disk 14 can be secured to the plate 12 utilizing a variety of methods. For instance, the inner disk 14 can have a plurality of protruding members (not show) which may be press fit into a matching plurality of openings 28 in the plate 12, and then sonic welded into position. Alternatively, the inner disk 14 may have a plurality of holes 30 which match the plurality of openings 28 in the plate 12. Rivets or set screws, for example, may be inserted through the plurality of holes 30 and into the openings 28 to secure the inner disk 14 to the plate 12, or the openings 28 may be internally threaded to receive standard screws. In another embodiment, a separate inner disk 14 component is not required, instead, a patterned section 24 is integral to the plate 12 itself.

The patterned section 24 of the inner disk 14 forms a releasable, interlocking coupling with a patterned portion 32 of the outer disk 16. The patterned section 24 and the patterned portion 32 can be a wide variety of patterns that cause interlocking to occur between the inner disk 14 and the outer disk 16. In one instance, the patterned portion 32 comprises a plurality of grooves 34, such as v-grooves, which releasably interlock with a mating plurality of grooves 26, such as v-grooves, in the patterned section 24 of the inner disk 14. In one particular embodiment, the plurality of grooves 34 and 26, in this case v-grooves, in the outer disk 16 and inner disk 14, respectively, are positioned in a radial pattern, wherein the v-grooves of the outer disk 16 are equally spaced 8 degrees apart such that 45 v-grooves exist in the patterned portion 32 of the outer disk 16 and the v-grooves of the inner disk 14 are equally spaced 8 degrees apart such that a mating 45 v-grooves exist in the patterned section 24 of the inner disk 14. Thus, enabling the adjustment device 10 to rotate 8 degrees, with respect to the accompanying orthopedic brace, per interlocking position between the inner disk 14 and the outer disk 16. This pattern can be produced, for example, by injection molding a material of 30% glass filled nylon, or another suitable material, or by machining grooves into the base material.

To secure the orthopedic brace to the adjustment device 10, a slot, such as a lateral slot, is produced in a section of the orthopedic brace, shown in FIG. 1 as slot 36 in orthopedic brace attachment arm 18. The outer disk 16 comprises a slot pin 33. The slot pin 33 slidably communicates with the slot 36 during lateral adjustments of the orthopedic brace with respect to the adjustment device 10. To facilitate these lateral adjustments, the slot pin 33 may be of a similar size and shape as the slot 36. In one embodiment, the patterned portion 32 is formed in the shape of the slot pin 33. As a result, the patterned portion functions as a slot pin, and two separate pieces are not required.

When the attachment arm 18 slot 36 is positioned adjacent to the plate opening 22, the patterned portion 32 of the outer disk 16 can be threaded through the slot 36 such that the patterned portion 32 of the outer disk 16 can be interlocked with the patterned section 24 of the inner disk 14. In one instance, the patterned portion 32 protrudes from the surface of the outer disk 16, enabling the patterned portion 32 to extend through the slot 36 and reach the patterned section 24 of the inner disk 14, allowing the inner disk 14 and the outer disk 16 to be engaged an interlocked coupling.

When the slot pin 33 of the outer disk 16 is placed within the slot 36 of the attachment arm 18, and the patterned portion 32 of the outer disk 16 is interlocked with the patterned section 24 of the inner disk 14, the attachment knob 20 can be secured to the inner disk 14. This, in turn, secures both the outer disk 16 to the attachment arm 18, and the attachment arm 18 to the plate 12. In one embodiment, the attachment knob 20 comprises a protruding member 38 having external threads, such as a screw. In this embodiment, the protruding member 38 extends through an opening 40 in the outer disk 16, through the slot 36 in the attachment arm 18 and into an internally threaded section 42 of the inner disk 14. In this arrangement, rotating the attachment knob 20 in a clockwise direction (assuming right handed threads) threads the protruding member 38 into the internally threaded section 42 of the inner disk 14. As a result, a compression force is produced, exerting a compression force on the outer disk 16, the attachment arm 18, and the plate 12. The compression force holds each of these components together.

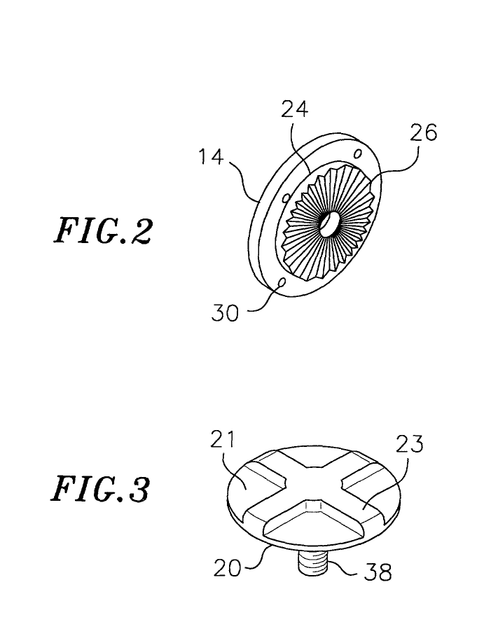

FIG. 3 shows a top surface 21 of the attachment knob 20. The top surface 21 of the attachment knob 20 comprises an ergonomic design 23. The ergonomic design 23 allows the attachment knob 20 to be both manually attached to the inner disk 14 and manually unattached to the inner disk 14 without the need for a tool. In one particular embodiment, the ergonomic design 23 is a section which protrudes from the top surface 21 of the attachment knob 20 in the shape of a cross, or the letter “X”. However, other ergonomic designs may be used.

Although the above discussion has focused on only the left side of the adjustment device 10, the descriptions used above are accurate for the right side of the adjustment device 10 as well and are omitted from the present discussion to avoid duplicity.

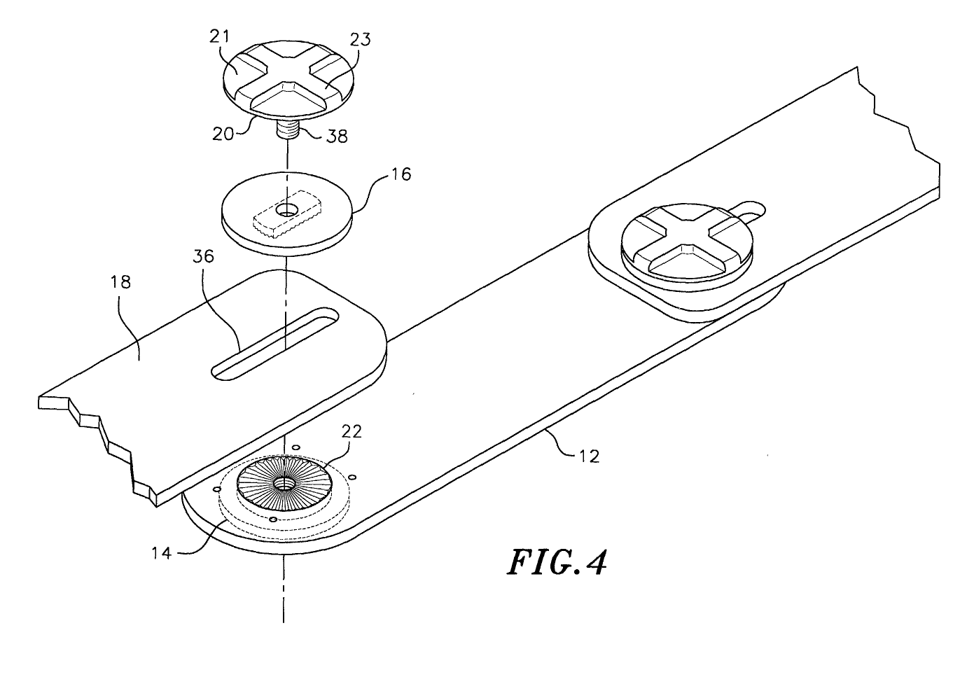

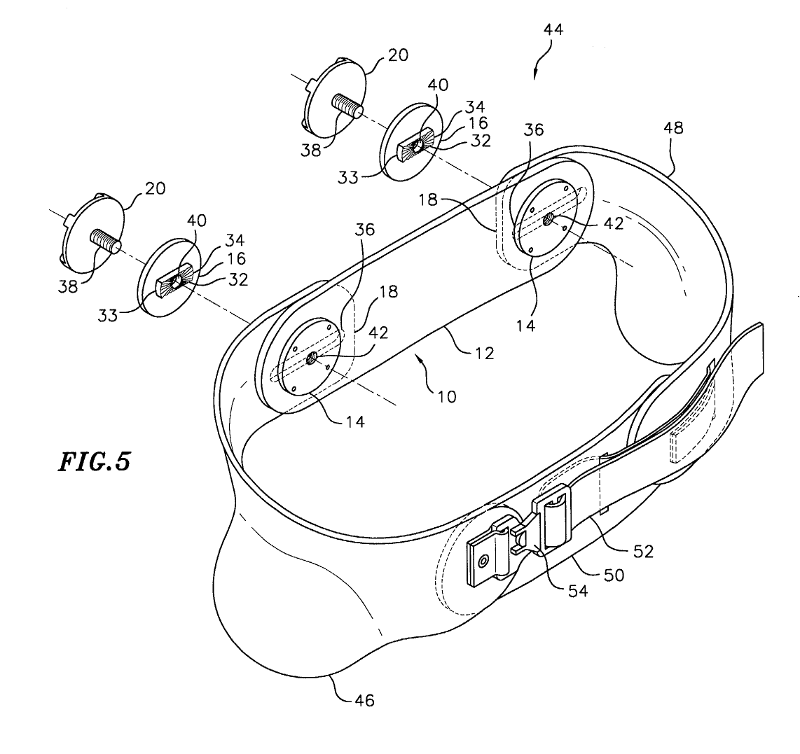

For illustrative purposes, FIG. 4 shows a partially assembled adjustment device 10. For visual clarity, the adjustment device 10 as shown is only attached to one orthopedic brace attachment arm 18.

FIG. 5 shows the adjustment device 10 of the present invention incorporated into an orthopedic hip brace 44. This brace 44 comprises a right hip shell 46, a left hip shell 48, a generally flexible front portion 50, an adjustment belt 52 and the adjustment device 10 of the current invention.

Typically, an orthopedic brace which incorporates an adjustment device, such as the adjustment device 10 of the current invention, is fitted to a user by a doctor or an orthopedic specialist. To custom fit the hip brace 44 to a user, the doctor typically lays the user flat on the user's back (in many cases the user has recently undergone hip surgery, therefore laying the user in any other position or standing the user is extremely painful to the user). The adjustment belt 52 can be detached from the hip brace 44 using a latch 54. Detaching the belt 52 allows the front portion 50 of the brace 44 to be opened. In the opened position, the brace 44 can be wrapped around the user's waist. The doctor can then place the hip shells 46 and 48 against the user's hips. Most likely, the doctor will then need to adjust the hip shells 46 and 48 both angularly and laterally in order to custom fit the brace 44 to the user. With the user still laying on his or her back, the doctor can reach behind the user, manually loosen the attachment knobs (right and left) 20 of the adjustment device 10, reposition the brace 44, and then manually tighten the attachment knobs 20. This operation requires only a minimum of movement by the user from the user's initial lying position. This manual adjustment of the attachment knobs 20 is an advantage over prior adjustment devices which require the use of an adjustment tool, such as an allen wrench, to loosen the adjustment device. Because an adjustment tool is required to adjust the adjustment device of the prior art, the doctor must move the user onto the user's side in order to fit the adjustment tool into the adjustment device. This is typically very painful to the user, especially one who has just undergone hip surgery.

After the attachment knobs 20 have been loosened, the shells 46 and 48 can be easily adjusted in the lateral direction by sliding the slot pin 33 of the outer disk 16 along the orthopedic brace attachment arm 18 slot 36 until a desired lateral position of each shell 46 and 48 is obtained, i.e. when the shell fits snugly against the user's hips in the lateral position. To adjust the shells 46 and 48 angularly, the outer disk 16 is disengaged from its interlocked coupling with the inner disk 14, and then rotated until a desired angular orientation is achieved. When the desired angular orientation is achieved, the outer disk 16 can be re-engaged to the inner disk 14 such that the disks 14 and 16 form an interlocking coupling. Since slot pin 33 is of a similar size and shape as the attachment arm 18 slot 36, rotating the outer disk 16 produces a rotational force that the slot pin 33 translates to the attachment arm 18 slot 36, thus angularly repositioning the orthopedic brace attachment arm 18 with respect to the adjustment device 10. An advantage that this arrangement provides is that once the brace 44 is adjusted to a desired lateral and angular position, the interlocking pattern of the patterned section 24 of the inner disk 14 and the patterned portion 32 of the outer disk 16 interlocks the disks 14 and 16 together such that the relative movement between the disks 14 and 16 is minimized. In addition, since the slot pin 33 is attached to the outer disk 16 and the slot pin 33 is of a similar size and shape of the attachment arm 18 slot 36, relative motion between the outer disk 16 and the orthopedic brace attachment arm 18 is minimized and thus relative motion between the adjustment device 10 and the orthopedic brace attachment arm 18 is minimized.

Although the present invention has been described and illustrated with respect to a preferred embodiment thereof, it is to be understood that the invention is not to be limited since changes and modifications are within the intended scope of the invention as hereinafter claimed.

1. An adjustment device for attachment to an orthopedic brace comprising:
a connecting plate;
an inner disk attached to the connecting plate, the inner disk having a patterned section; and
an outer disk having a patterned portion which forms an interlocking coupling with the patterned section of the inner disk to releasably interlock the inner disk to the outer disk.

2. The adjustment device of claim 1 further comprising:
an attachment knob having an extending member, wherein the attachment knob applies a compressive force to the outer disk to secure the outer disk to the inner disk when the extending member is mated with the inner disk.

3. The adjustment device of claim 2 wherein the outer disk mates with an orthopedic brace attachment arm having a slot, the outer disk slidably communicates with the attachment arm slot to allow for a lateral adjustment of the orthopedic brace.

4. The adjustment device of claim 3, wherein the outer disk may be disengaged from the inner disk and rotated with respect to the inner disk to cause the attachment arm to rotate with respect to the connecting plate when the outer disk is mated with the attachment arm slot.

5. The adjustment device of claim 4, wherein the outer disk comprises a protruding section which is substantially similar to the shape of the attachment arm slot such that the protruding section mates with the attachment arm slot.

6. The adjustment device of claim 5, wherein the patterned section of the inner disk and the patterned portion of the outer disk each comprise a plurality of v-grooves arranged in a radial pattern wherein a center line of each v-groove is radially spaced a same number of degrees from an adjacent v-groove centerline.

7. The adjustment device of claim 6, wherein the adjustment knob comprises an ergonomic design such that the knob can be manually attached to and detached from the inner disk without the use of tools.

8. The adjustment device of claim 7, wherein the orthopedic brace is an orthopedic hip brace.

9. An adjustment device for attachment to an orthopedic brace comprising:
a connecting plate having an opening;
an inner disk having a patterned raised section which mates with the connecting plate opening; and
an outer disk having a patterned protruding portion which forms an interlocking coupling with the patterned raised section of the inner disk to releasably interlock the inner disk to the outer disk.

10. The adjustment device of claim 9 further comprising:
an attachment knob having an externally threaded extending member, wherein the attachment knob applies a compressive force to the outer disk to secure the outer disk to the inner disk when the externally threaded extending member is mated with an internally threaded section of the inner disk.

11. The adjustment device of claim 10 wherein the protruding portion of the outer disk mates with an orthopedic brace attachment arm having a slot, the protruding portion having a shape which is substantially similar to the shape of the attachment arm slot such that the protruding portion mates with the attachment arm slot.

12. The adjustment device of claim 11, wherein the protruding portion mates with the attachment arm slot to allow for a lateral adjustment of the orthopedic brace when the attachment arm is disposed between the connecting plate and the outer disk.

13. The adjustment device of claim 12, wherein rotating the outer disk with respect to the inner disk causes the orthopedic brace attachment arm to rotate with respect to the connecting plate when the outer disk is mated with the orthopedic brace attachment arm slot.

14. The adjustment device of claim 13, wherein the patterned raised section of the inner disk and the patterned protruding portion of the outer disk each comprise a plurality of v-grooves arranged in a radial pattern wherein a center line of each v-groove is radially spaced a same number of degrees from an adjacent v-groove centerline.

15. The adjustment device of claim 14, wherein the adjustment knob comprises an ergonomic design such that the knob can be manually attached to and detached from the inner disk without the use of tools.

16. An adjustment device for attachment to an orthopedic hip brace comprising:
a connecting plate having a first opening and a second opening;
a first inner disk having a patterned raised section which mates with the connecting plate first opening;
a second inner disk having a patterned raised section which mates with the connecting plate second opening;
a first outer disk having a patterned protruding portion which forms an interlocking coupling with the patterned raised section of the inner disk to releasably interlock the inner disk to the outer disk; and
a second outer disk having a patterned protruding portion which forms an interlocking coupling with the patterned raised section of the inner disk to releasably interlock the inner disk to the outer disk.

17. The adjustment device of claim 16 further comprising:
a first attachment knob having an externally threaded extending member, wherein the first attachment knob applies a compressive force to the first outer disk to secure the first outer disk to the first inner disk when the externally threaded extending member of the first attachment knob is mated with an internally threaded section of the first inner disk; and
a second attachment knob having an externally threaded extending member, wherein the second attachment knob applies a compressive force to the second outer disk to secure the second outer disk to the second inner disk when the externally threaded extending member of the second attachment knob is mated with an internally threaded section of the second inner disk.

18. The adjustment device of claim 17 wherein:
the protruding portion of the first outer disk mates with a first orthopedic hip brace attachment arm having a slot, the protruding portion of the first outer disk having a shape which is substantially similar to the shape of the first attachment arm slot such that the protruding portion of the first outer disks mates with the first attachment arm slot to allow for a first lateral adjustment of the orthopedic hip brace when the first attachment arm is disposed between the connecting plate and the first outer disk; and
the protruding portion of the second outer disk mates with a second orthopedic hip brace attachment arm having a slot, the protruding portion of the second outer disk having a shape which is substantially similar to the shape of the second attachment arm slot such that the protruding portion of the second outer disks mates with the second attachment arm slot to allow for a second lateral adjustment of the orthopedic hip brace when the second attachment arm is disposed between the connecting plate and the second outer disk.

19. The adjustment device of claim 18, wherein:
rotating the first outer disk with respect to the first inner disk causes the orthopedic hip brace attachment arm to rotate with respect to the connecting plate when the first outer disk is mated with the orthopedic hip brace attachment arm slot; and
rotating the second outer disk with respect to the second inner disk causes the orthopedic hip brace attachment arm to rotate with respect to the connecting plate when the second outer disk is mated with the orthopedic hip brace attachment arm slot.

20. The adjustment device of claim 19, wherein the patterned raised section of the inner disk and the patterned protruding portion of the outer disk each comprise a plurality of v-grooves arranged in a radial pattern wherein a center line of each v-groove is radially spaced a same number of degrees from an adjacent v-groove centerline.
